(19) United States
(12) Patent Application Publication
(10) Pub. No.: US 2018/0055496 A1
(43) Pub. Date: Mar. 1, 2018

(54) JUGULAR ACCESS LEFT ATRIAL APPENDAGE CLOSURE DEVICE

(71) Applicant: CARDIAC PACEMAKERS, INC., St. Paul, MN (US)

(72) Inventors: Dongming Hou, Plymouth, MN (US); Hong Cao, Maple Grove, MN (US)

(73) Assignee: CARDIAC PACEMAKERS, INC., St. Paul, MN (US)

(21) Appl. No.: 15/684,157

(22) Filed: Aug. 23, 2017

Related U.S. Application Data
(60) Provisional application No. 62/378,479, filed on Aug. 23, 2016.

(51) Int. Cl. A61B 17/00; A61B 17/12

(52) U.S. Cl. CPC A61B 17/0057; A61B 2017/00243; A61B 17/12013

(57) ABSTRACT

A left atrial appendage closure device system may include an access sheath having a lumen, a delivery sheath slidably disposed within the lumen of the access sheath, and a left atrial appendage closure device slidably disposed within a distal portion of the delivery sheath. The access sheath may include a pre-curved portion adjacent a distal tip of the access sheath. A method of deploying a left atrial appendage closure device may include advancing an access sheath through the superior vena cava and into the right atrium of the heart, advancing a delivery sheath through the access sheath into the left atrium, and deploying the left atrial appendage closure device within the ostium of the left atrial appendage.

### CROSS-REFERENCE TO RELATED APPLICATIONS

This application claims the benefit of and priority to U.S. Provisional Application No. 62/378,479 filed Aug. 23, 2016 the entire contents of which are herein incorporated by reference.

### TECHNICAL FIELD

The present disclosure pertains to percutaneous medical devices and methods for using percutaneous medical devices. More particularly, the present disclosure pertains to percutaneous medical devices for implantation into the left atrial appendage (LAA) of a heart.

### BACKGROUND

A wide variety of intracorporeal medical devices have been developed for medical use, for example, surgical and/or intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and/or using medical devices.

### SUMMARY

In a first aspect, a left atrial appendage closure device system may comprise an access sheath having a lumen extending therethrough, a delivery sheath slidably disposed within the lumen of the access sheath, and a left atrial appendage closure device slidably disposed within a distal portion of the delivery sheath. The access sheath may include a pre-curved portion adjacent a distal tip of the access sheath. The pre-curved portion may have a bend radius between about 2.25 inches and about 4.00 inches.

In addition or alternatively, and in a second aspect, the access sheath includes a body portion having a length between about 15 inches and about 20 inches.

In addition or alternatively, and in a third aspect, wherein the access sheath includes a proximal hub.

In addition or alternatively, and in a fourth aspect, the body portion extends from the proximal hub to the pre-curved portion.

In addition or alternatively, and in a fifth aspect, the pre-curved portion forms a tip angle of between about 110 degrees and about 140 degrees.

In addition or alternatively, and in a sixth aspect, the pre-curved portion forms a tip angle of about 125 degrees.

In addition or alternatively, and in a seventh aspect, the bend radius is about 3.00 inches.

In addition or alternatively, and in an eighth aspect, the length of the body portion is about 17.72 inches.

In addition or alternatively, and in a ninth aspect, the access sheath has an outer diameter between about 10 F. and about 18 F.

In addition or alternatively, and in a tenth aspect, the access sheath has an outer diameter of about 14 F.

In addition or alternatively, and in an eleventh aspect, the delivery sheath has an outer diameter about 2 F smaller than an outer diameter of the access sheath.

In addition or alternatively, and in a twelfth aspect, the delivery sheath has an outer diameter of about 12 F.

In addition or alternatively, and in a thirteenth aspect, the left atrial appendage closure device is actuatable between a collapsed configuration and an expanded configuration.

In addition or alternatively, and in a fourteenth aspect, the left atrial appendage closure device is disposed within the delivery sheath in the collapsed configuration.

In addition or alternatively, and in a fifteenth aspect, the left atrial appendage closure device is releasably attached to a core wire slidably disposed within the delivery sheath.

In addition or alternatively, and in a sixteenth aspect, a method of deploying a left atrial appendage closure device in a heart may include advancing an access sheath through a superior vena cava to a right atrium of the heart, advancing a delivery sheath through the access sheath and into a left atrium of the heart, and deploying a left atrial appendage closure device within an ostium of a left atrial appendage.

In addition or alternatively, and in a seventeenth aspect, advancing the access sheath through the superior vena cava to the right atrium of the heart positions the access sheath through a wall of the heart between the right atrium and the left atrium and a distal tip of the access sheath proximate the ostium of the left atrial appendage.

In addition or alternatively, and in an eighteenth aspect, advancing the delivery sheath through the access sheath and into the left atrium of the heart includes positioning a distal end of the delivery sheath within the ostium of the left atrial appendage.

In addition or alternatively, and in a nineteenth aspect, the access sheath includes a pre-curved portion adjacent the distal tip of the access sheath, the pre-curved portion having a bend radius of about 3.00 inches.

In addition or alternatively, and in a twentieth aspect, a method of deploying a left atrial appendage closure device may include advancing a distal tip of an access sheath within a superior vena cava and into a right atrium of a heart, positioning the distal tip of the access sheath within a left atrium of the heart proximate an ostium of a left atrial appendage, advancing a delivery sheath slidably disposed within the access sheath into the left atrium, deploying a left atrial appendage closure device within the ostium of the left atrial appendage of the heart, the left atrial appendage closure device being releasably attached to a core wire slidably disposed within the delivery sheath, expanding the left atrial appendage closure device into engagement with the ostium of the left atrial appendage, and detaching the left atrial appendage closure device from the core wire.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a partial cut-away view of an example heart;

FIGS. 2 and 3 illustrate a prior art approach to the left atrial appendage;

FIG. 4 illustrates a prior art access sheath for the approach of FIGS. 2 and 3;

FIGS. 5 and 6 illustrate an example approach to the left atrial appendage;

FIG. 7 illustrates an example access sheath for use with the approach of FIGS. 5 and 6;

FIG. 8A is an end view showing an example configuration of the access sheath of FIG. 7; and

FIG. 8B is an end view showing an example configuration of the access sheath of FIG. 7.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the claimed invention. Those skilled in the art will recognize that the various elements described and/or shown may be arranged in various combinations and configurations without departing from the scope of the disclosure. The detailed description and drawings illustrate example embodiments of the claimed invention.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g. 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosed invention are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Atrial fibrillation (AF) is a common cardiac arrhythmia affecting over 5.5 million people worldwide. Atrial fibrillation is the irregular, chaotic beating of the upper chambers of the heart. Electrical impulses discharge so rapidly that the atrial muscle quivers, or fibrillates. Episodes of atrial fibrillation may last a few minutes or several days. The most serious consequence of atrial fibrillation is ischemic stroke. It has been estimated that up to 20% of all strokes are related to atrial fibrillation. Most atrial fibrillation patients, regardless of the severity of their symptoms or frequency of episodes, require treatment to reduce the risk of stroke. The left atrial appendage (LAA) is a small organ attached to the left atrium of the heart as a pouch-like extension. In patients suffering from atrial fibrillation, the left atrial appendage may not properly contract with the left atrium, causing stagnant blood to pool within its interior, which can lead to the undesirable formation of thrombi within the left atrial appendage. Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation are found in the left atrial appendage. As a treatment, medical devices have been developed which are positioned in the left atrial appendage and deployed to close off the ostium of the left atrial appendage. Over time, the exposed surface(s) spanning the ostium of the left atrial appendage becomes covered with tissue (a process called endothelization), effectively removing the left atrial appendage from the circulatory system and reducing or eliminating the amount of thrombi which may enter the blood stream from the left atrial appendage.

Disclosed herein are apparatus, medical devices, and/or methods that may be used for removing the left atrial appendage from the circulatory system and reducing or eliminating the amount of thrombi which may enter the blood stream from the left atrial appendage. At least some of the apparatus, medical devices, and/or methods disclosed herein may include and/or be used to deliver and implant a left atrial appendage closure device using minimally-invasive intravascular techniques. While access via the trans-femoral vein is commonly used in some techniques, cultural and/or other medical reasons (e.g., inferior vena cava obstruction, anatomical abnormality, etc.) may warrant a different approach. The devices and methods disclosed herein may also provide a number of additional desirable features and/or benefits as described in more detail below.

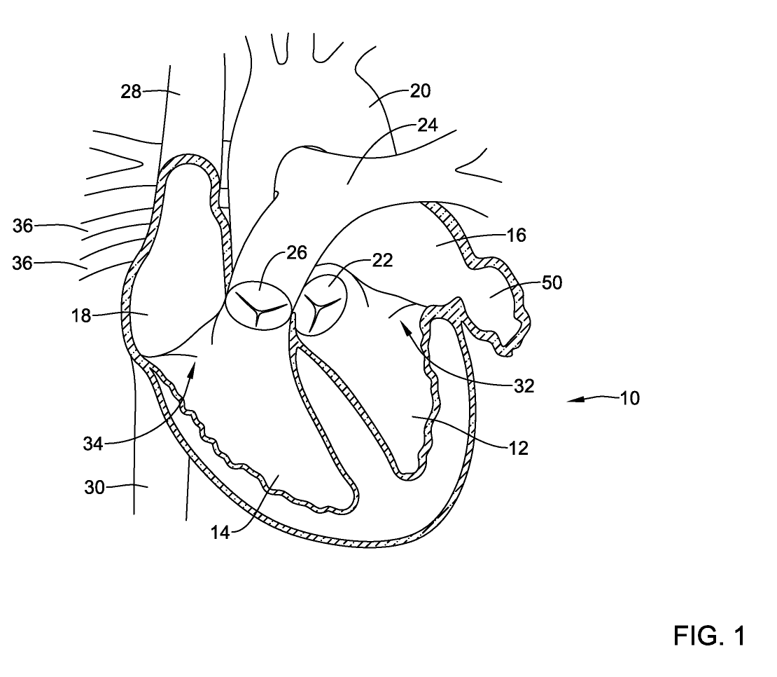

FIG. 1 is a partial cross-sectional view of certain elements of a heart 10 and some immediately adjacent blood vessels. A heart 10 may include a left ventricle 12, a right ventricle 14, a left atrium 16, and a right atrium 18. An aortic valve 22 is disposed between the left ventricle 12 and an aorta 20. A pulmonary or semi-lunar valve 26 is disposed between the right ventricle 14 and a pulmonary artery 24. A superior vena cava 28 and an inferior vena cava 30 return blood from the body to the right atrium 18. A mitral valve 32 is disposed between the left atrium 16 and the left ventricle 12. A tricuspid valve 34 is disposed between the right atrium 18 and the right ventricle 14. Pulmonary veins 36 return blood from the lungs to the left atrium 16. A left atrial appendage (LAA) 50 is attached to and in fluid communication with the left atrium 16.

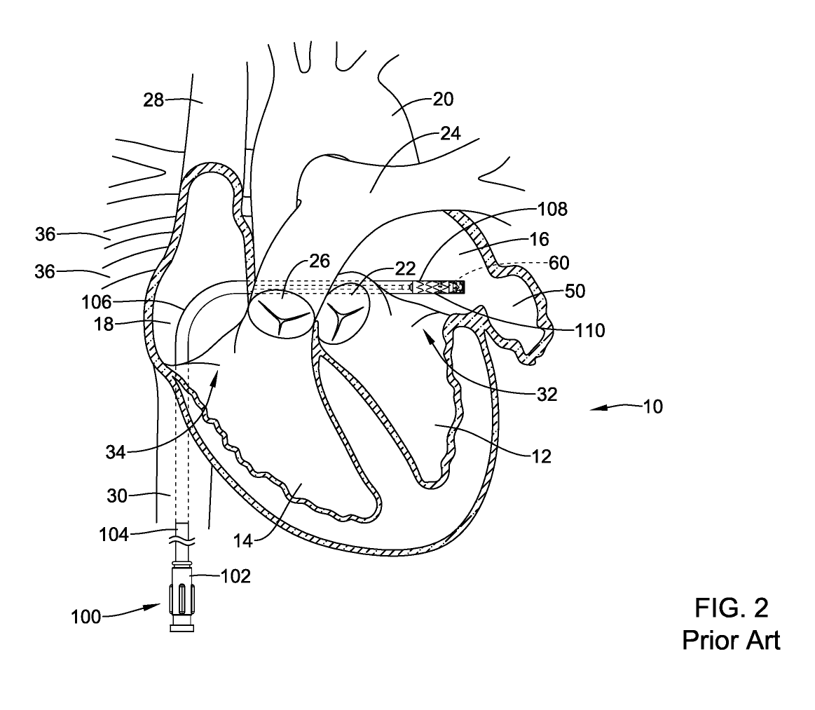

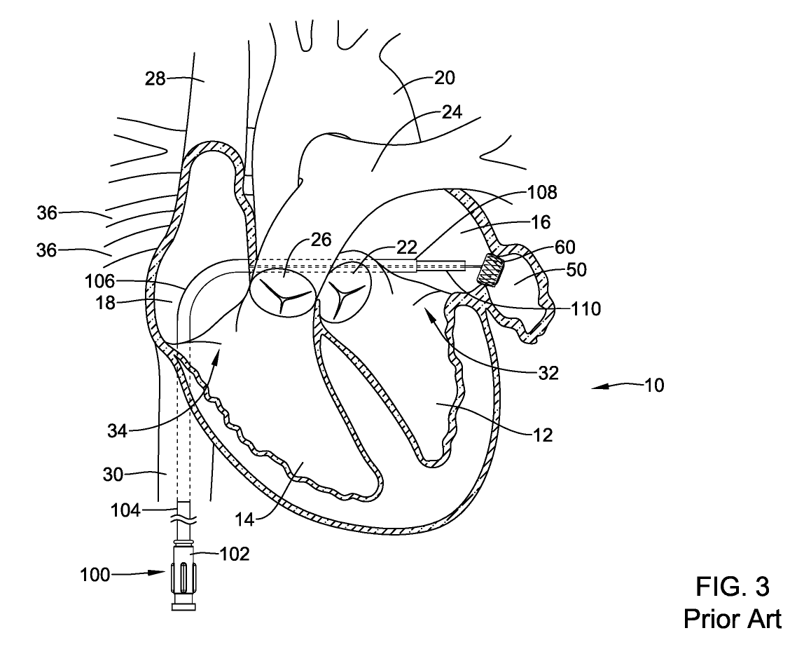

FIGS. 2-3 generally illustrate a prior art approach to the left atrial appendage 50. A prior art access sheath 100 is guided toward the heart 10 via the inferior vena cava 30 to the right atrium 18. As viewed from an anterior side of a patient, a distal portion of the prior art access sheath 100 may include a right-hand curve near its distal end, such that the prior art access sheath 100 curves toward a wall (e.g., the septum) between the right atrium 18 and the left atrium 16. The wall between the right atrium 18 and the left atrium 16 is punctured and a delivery sheath 110 is extended from the prior art access sheath 100 and through the wall to a position adjacent the left atrial appendage 50. A left atrial appendage closure device 60 may be delivered into the left atrial appendage 50 from the delivery sheath 110, as seen in FIG. 3 for example.

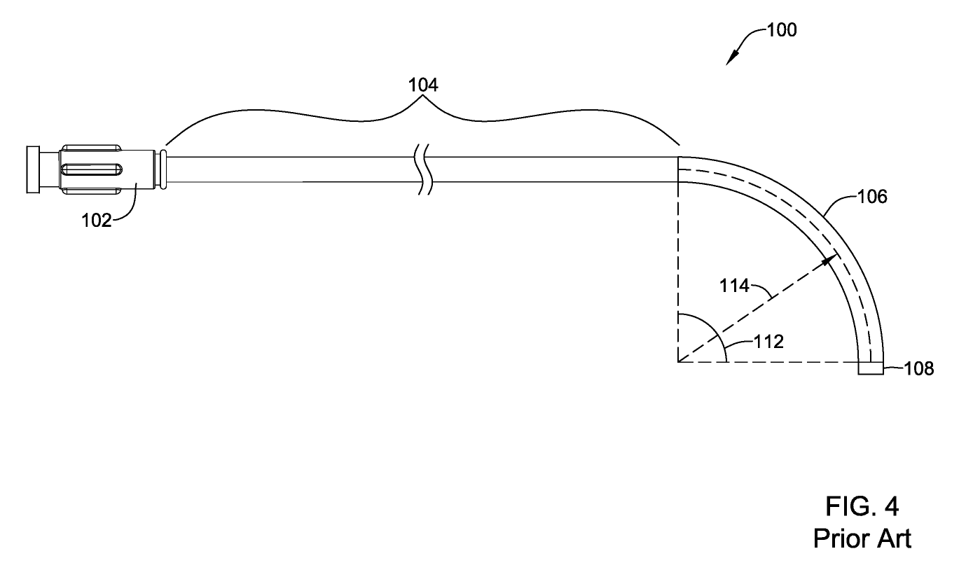

FIG. 4 generally illustrates a prior art access sheath 100. The prior art access sheath 100 may include a proximal hub 102 and a body portion 104 extending from the proximal hub 102 to a distal tip 108. A pre-curved portion 106 of the body portion 104 may be disposed near the distal tip 108. The pre-curved portion 106 has a bend radius 114 of about 2.125 inches (about 5.4 cm) and the pre-curved portion 106 curves or bends the body portion 104 about 90 degrees. In other words, a tip angle 112 (e.g., the angle between the body portion 104 and the distal tip 108) is about 90 degrees. The body portion 104 extends about 27.56 inches (about 70 cm) from the proximal hub 102 to the pre-curved portion 106. The pre-curved portion 106 of the prior art access sheath 100 comes in three configurations—single curve, double curve, and anterior curve.

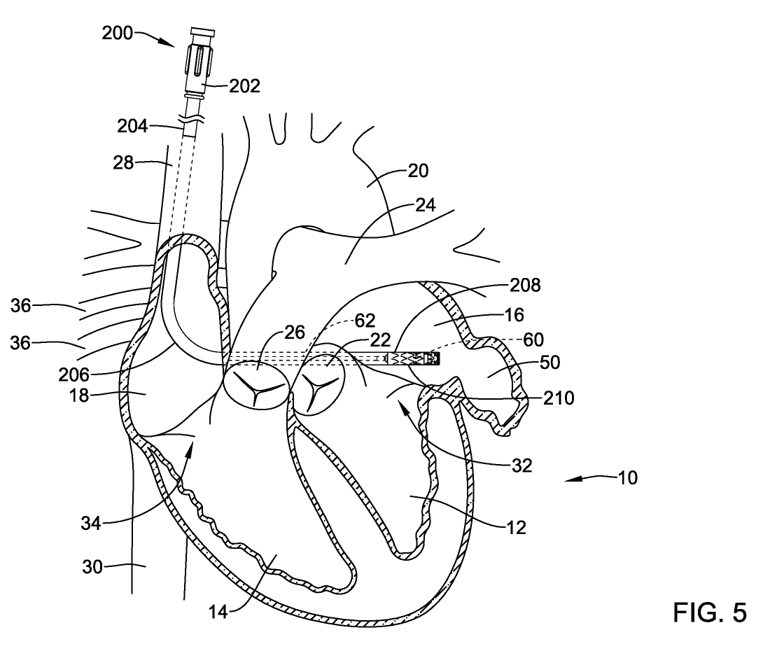

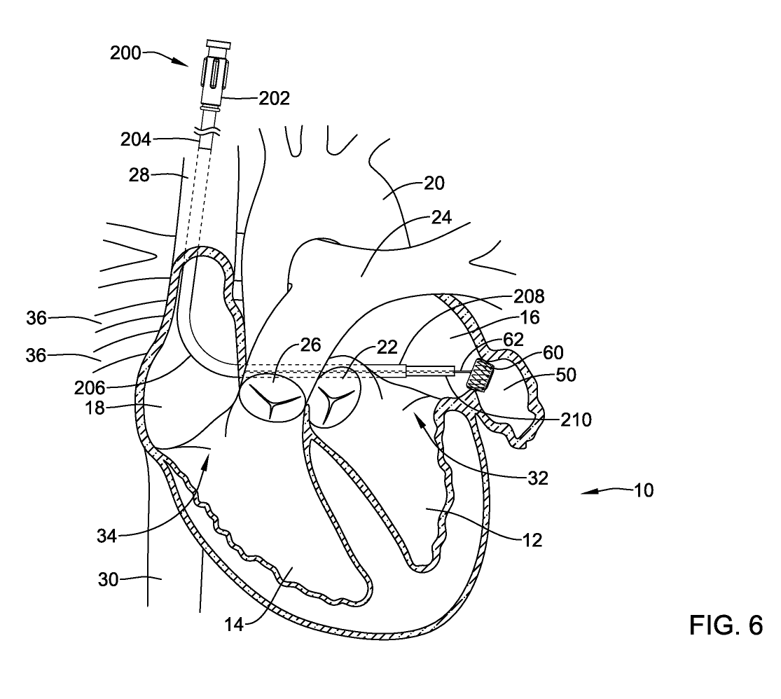

FIGS. 5-6 illustrate an example method of using a left atrial appendage closure device system to deploy a left atrial appendage closure device 60 using an access sheath 200 adapted and configured to access the right atrium 18 via the superior vena cava 28, a jugular vein, and/or another suitable vessel in fluid communication with the superior vena cava 28. The method will be explained in more detail below. In order to better understand the method, a description of an example access sheath 200 is now provided.

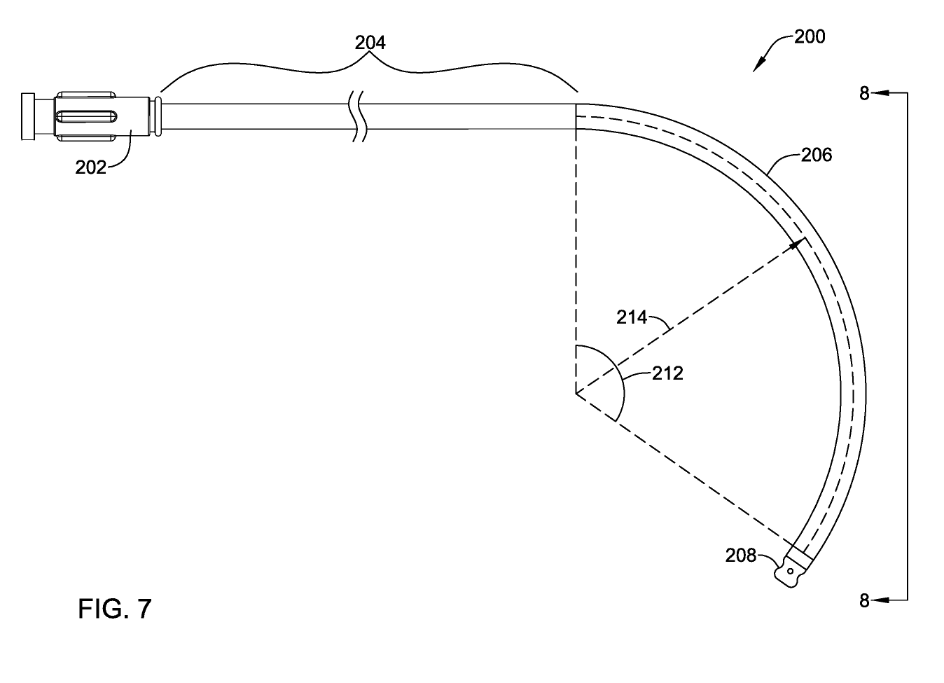

The access sheath 200 may be an elongated tubular member having a proximal hub 202, a body portion 204 extending from the proximal hub 202 to a pre-curved portion 206, and the pre-curved portion 206 extending from the body portion 204 to a distal tip 208, as seen in FIG. 7 for example. The orientation of the access sheath 200 as shown in FIG. 7, if the access sheath 200 were positioned within superior vena cava 28 and the heart 10 of a patient, corresponds to a posterior view of the access sheath 200, or looking in the anterior direction from behind the patient.

The pre-curved portion 206 of the access sheath 200 may be disposed adjacent the distal tip 208. In some embodiments, the pre-curved portion 206 may be heat set, formed from a shape memory material, or other suitable means to attain a predetermined and predisposed configuration. In other words, when in an unstressed condition, the access sheath 200 may be self-biased toward the predetermined and predisposed arrangement, including the pre-curved portion 206 being curved or bent relative to the body portion 204. The pre-curved portion 206 may have a bend radius 214 of about 3.00 inches (about 7.62 cm). In some embodiments, the pre-curved portion 206 may have a bend radius 214 of about 2.25 inches, 2.50 inches, 2.75 inches, 3.25 inches, 3.5 inches, 3.75 inches, or another suitable radius. The pre-curved portion 206 may curve or bend the body portion 204 between about 90 degrees and about 150 degrees, between about 110 degrees and about 140 degrees, about 125 degrees, or another suitable angle. In other words, a tip angle 212 (e.g., the angle between the body portion 204 and the distal tip 208) measured at and/or relative to the center of the bend radius is between about 90 degrees and about 150 degrees, between about 105 degrees and about 135 degrees, about 125 degrees, or another suitable angle.

The body portion 204 may extend about 17.72 inches (about 45 cm) from the proximal hub 202 to the pre-curved portion 206. In some embodiments, the body portion 204 may extend between about 10 inches and about 30 inches, between about 12 inches and about 25 inches, between about 14 inches and about 20 inches, about 17.72 inches, or another suitable length from the proximal hub 202 to the pre-curved portion 206. In some embodiments, the access sheath 200, the body portion 204, the pre-curved portion 206, and/or the distal tip 208 may have an outer diameter or extent of about 18 F (French), about 16 F, about 14 F, about 12 F, about 10 F, about 8 F, or another suitable measurement. In some embodiments, the access sheath 200 may be tapered from a first diameter at the proximal hub 202 to a second smaller diameter at the body portion 204, the pre-curved portion 206, and/or the distal tip 208. In some embodiments, the second smaller diameter may be about 14 F, or another suitable measurement.

In some embodiments, the distal tip 208 may be a soft distal tip and may include a plurality of vent holes spaced around the circumference of the distal tip 208 (e.g., 3 holes at 60 degree angles from each other, 4 holes at 90 degree angles from each other, etc.). The soft distal tip may be atraumatic to tissues and/or walls of the superior vena cava 28, the heart 10, and/or elements thereof. In some embodiments, the plurality of vent holes may facilitate flexing of the soft distal tip and/or may reduce pressure if/when injecting contrast fluid. In some embodiments, the plurality of holes may each have a diameter of between about 0.02225 inches (0.5652 mm) and about 0.050 inches (1.270 mm), or between about 24 gauge and about 18 gauge. Other sizes and/or configurations for the plurality of holes are also contemplated.

Turning back to FIG. 5, the access sheath 200 may be advanced percutaneously toward the heart 10 via the superior vena cava 28 to the right atrium 18. Accessing the heart 10 via the superior vena cava 28 may permit the use of a shorter length access sheath 200, may reduce patient discomfort and/or procedure time, and/or may avoid some cultural difficulties or objections. Other advantages and/or benefits will be apparent to the skilled practitioner. As viewed from an anterior side of a patient, a distal portion of the access sheath 200 may include a left-hand curve near its distal tip 208 (e.g., at the pre-curved portion 206), such that the access sheath 200 curves toward a wall (e.g., the septum) between the right atrium 18 and the left atrium 16. The wall between the right atrium 18 and the left atrium 16 may be punctured, using a suitable structure and/or method, and the distal tip 208 of the access sheath 200 may be extended through the wall between the right atrium 18 and the left atrium 16 to a position within the left atrium 16 proximate an ostium of the left atrial appendage 50. In some embodiments, a dilator (not shown) may be inserted through and/or along with the access sheath 200 to gain access to the left atrium 16 and/or the left atrial appendage 50 after transseptal access into the left atrium 16 has been established.

The delivery sheath 210 may be sized and configured to be slidably disposed and/or received within a lumen of the access sheath 200. In some embodiments, the delivery sheath 210 may have an outer diameter or extent at and/or near a distal end of the delivery sheath 210 of about 16 F (French), about 14 F, about 12 F, about 10 F, about 8 F, about 6 F, or another suitable measurement. In some embodiments, the delivery sheath 210 may be tapered from a first diameter at a proximal end to a second smaller diameter at a distal end. In some embodiments, the second smaller diameter may be about 12 F, or another suitable measurement. In some embodiments, an outer diameter or extent of the delivery sheath 210 may be about 2 F smaller than the access sheath 200.

A left atrial appendage closure device 60 may include a support frame configured to actuate between a collapsed configuration and an expanded configuration. In some embodiments, the support frame may be configured to reversibly actuate between the collapsed configuration and the expanded configuration. In some embodiments, the support frame may include a plurality of struts and/or a stent-like structure. In some embodiments, the left atrial appendage closure device 60 and/or the support frame may be sized and/or configured to be disposed within and/or to occlude the ostium of the left atrial appendage 50. In some embodiments, the left atrial appendage closure device 60 may include an occlusive element (e.g., a fabric, a mesh, a membrane, etc.) disposed on, disposed over, disposed about, and/or covering at least a portion of the support frame. In some embodiments, the left atrial appendage closure device 60 may include a coupling structure configured to releasably attach the left atrial appendage closure device 60 to a distal end of a core wire 62.

The left atrial appendage closure device 60 may be slidably disposed within a distal portion of a lumen of the delivery sheath 210 in the collapsed configuration, wherein the support frame fits within the lumen of the delivery sheath 210, as seen in FIG. 5 for example. The core wire 62 may extend proximally from the left atrial appendage closure device 60 within the lumen of the delivery sheath 210. The core wire 62 may be manually and/or mechanically manipulatable outside of the delivery sheath 210, the access sheath 200, and/or the patient.

The delivery sheath 210 may be advanced through the access sheath 200 and into the left atrium 16 of the heart 10 and a distal end of the delivery sheath 210 positioned proximate the ostium of the left atrial appendage 50. In embodiments using a dilator, the dilator is first removed from the access sheath 200 while the access sheath 200 is held in a fixed position with the distal tip 208 proximate the ostium of the left atrial appendage 50 before the delivery sheath 210 is introduced into the access sheath 200. In some embodiments, the delivery sheath 210 may be fixed into position within and relative to the access sheath 200, for example, using a snap-fit, a threaded connection, a locking collar, a set screw, a pin, a spring, or other suitable fixing arrangement.

After positioning the delivery sheath 210 within the left atrium 16, the left atrial appendage closure device 60 may be deployed from the distal end of the delivery sheath 210, as seen in FIG. 6 for example. In at least some embodiments, the left atrial appendage closure device 60 may be delivered into the ostium of the left atrial appendage 50 from the delivery sheath 210. In some embodiments, the left atrial appendage closure device 60 may be advanced distally relative to and/or out of the delivery sheath 210. In some embodiments, the delivery sheath 210 may be advanced to a position at or in the ostium of the left atrial appendage 50, the core wire 62 may be held in a fixed position, and the delivery sheath 210 may be withdrawn relative to the left atrial appendage closure device 60, thereby deploying the left atrial appendage closure device 60 at or within the ostium of the left atrial appendage 50.

Upon or after deploying the left atrial appendage closure device 60 from the delivery sheath 210, the left atrial appendage closure device 60 may expand and/or be actuated to the expanded configuration. In the expanded configuration, the left atrial appendage closure device 60 may come into contact with and/or sealingly engage the ostium of the left atrial appendage 50. In some embodiments, if the positioning of the left atrial appendage closure device 60 needs to be adjusted, the left atrial appendage closure device 60 may be actuated back toward and/or to the collapsed configuration to facilitate repositioning the left atrial appendage closure device 60. Once placement of the left atrial appendage closure device 60 is determined to be satisfactory, the coupling structure may release the left atrial appendage closure device 60 from the distal end of the core wire 62. After release of the left atrial appendage closure device 60, the core wire 62, the delivery sheath 210, and/or the access sheath 200 may be withdrawn from the patient's vasculature and appropriate sealing and/or clotting procedures initiated.

The access sheath 200 shown in FIG. 7 may be configured in one of several possible configurations, wherein the style or type of curve at the pre-curved portion 206 may be varied to assist with placement of the access sheath 200 according to the patient's anatomy, physician's preference, etc. In some embodiments, the access sheath 200 may have or include a “single curve” configuration. In some embodiments, the access sheath 200 may have or include a “double curve” configuration. In some embodiments, the access sheath 200 may have or include an “anterior curve” configuration. Other configurations are also contemplated.

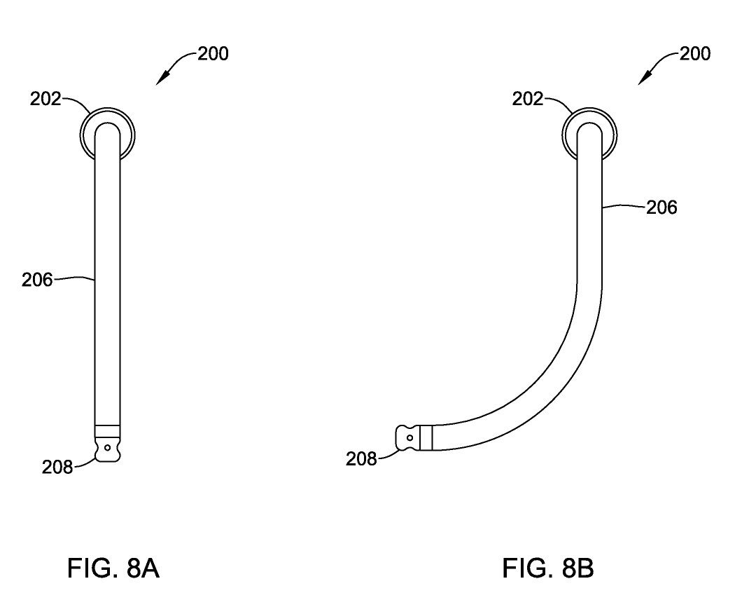

FIG. 8A illustrates an end view of an example configuration of the access sheath 200 shown in FIG. 7. The configuration shown in FIG. 8A may be considered a “single curve” configuration, wherein the pre-curved portion 206 of the access sheath 200 curves in one direction within a single plane. In at least some embodiments, the pre-curved portion 206 of the access sheath 200 may curve within and/or along a coronal plane when positioned within the heart of a patient.

FIG. 8B illustrates an end view of an example configuration of the access sheath 200 shown in FIG. 7. The configuration shown in FIG. 8B may be considered a “double curve” configuration, wherein the pre-curved portion 206 of the access sheath 200 curves in two directions within two planes. In at least some embodiments, the pre-curved portion 206 of the access sheath 200 may curve within and/or along a coronal plane when positioned within the heart of a patient and again at, within, and/or along a transverse plane. Generally speaking, a length of the curvature of the “double curve” configuration of the pre-curved portion 206 may be about the same as the “single curve” configuration. The “double curve” configuration may include a second curve beginning at about one-third to one-half of the tip angle 212 from the distal tip 208, wherein the second curve may bend between about 10 degrees and about 45 degrees, between about 15 degrees and about 30 degrees, etc. at, within, and/or along a transverse plane in an anterior direction (e.g., toward the front and/or away from the coronal plane of the patient).

The materials that can be used for the various components of the access sheath 200, the delivery sheath 210, the core wire 62, the left atrial appendage closure device 60, etc. (and/or other systems disclosed herein) and the various elements thereof disclosed herein may include those commonly associated with medical devices. For simplicity purposes, the following discussion makes reference to the access sheath 200, the delivery sheath 210, the core wire 62, the left atrial appendage closure device 60, etc. However, this is not intended to limit the devices and methods described herein, as the discussion may be applied to other elements, members, components, or devices disclosed herein, such as, but not limited to, the access sheath 200, the delivery sheath 210, the core wire 62, the left atrial appendage closure device 60, etc. and/or elements or components thereof.

In some embodiments, the access sheath 200, the delivery sheath 210, the core wire 62, the left atrial appendage closure device 60, etc., and/or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the access sheath 200, the delivery sheath 210, the core wire 62, the left atrial appendage closure device 60, etc., and/or components thereof, may also be doped with, made of, or otherwise include a radiopaque material and/or discrete radiopaque markers. Radiopaque materials and/or markers are understood to be capable of producing a relatively bright image on a fluoroscopy screen or another imaging technique during a medical procedure. This relatively bright image aids a user in determining the location of the access sheath 200, the delivery sheath 210, the core wire 62, the left atrial appendage closure device 60, etc. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the access sheath 200, the delivery sheath 210, the core wire 62, the left atrial appendage closure device 60, etc. to achieve the same result.

In some embodiments, a degree of Magnetic Resonance Imaging (MRI) compatibility is imparted into the access sheath 200, the delivery sheath 210, the core wire 62, the left atrial appendage closure device 60, etc. For example, the access sheath 200, the delivery sheath 210, the core wire 62, the left atrial appendage closure device 60, etc., and/or components or portions thereof, may be made of a material that does not substantially distort the image and create substantial artifacts (e.g., gaps in the image). Certain ferromagnetic materials, for example, may not be suitable because they may create artifacts in an Mill image. The access sheath 200, the delivery sheath 210, the core wire 62, the left atrial appendage closure device 60, etc., or portions thereof, may also be made from a material that the MRI machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

In some embodiments, the access sheath 200, the delivery sheath 210, the core wire 62, the left atrial appendage closure device 60, etc., and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the left atrial appendage closure device 60 may include a fabric material and/or a membrane disposed over or within the support frame. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. A left atrial appendage closure device system, comprising:
an access sheath having a lumen extending therethrough;
a delivery sheath slidably disposed within the lumen of the access sheath; and
a left atrial appendage closure device slidably disposed within a distal portion of the delivery sheath;
wherein the access sheath includes a pre-curved portion adjacent a distal tip of the access sheath, the pre-curved portion having a bend radius between about 2.10 inches and about 4.00 inches.

2. The left atrial appendage closure device system of claim 1, wherein the access sheath includes a body portion having a length between about 15 inches and about 5 inches.

3. The left atrial appendage closure device system of claim 2, wherein the access sheath includes a proximal hub.

4. The left atrial appendage closure device system of claim 3, wherein the body portion extends from the proximal hub to the pre-curved portion.

5. The left atrial appendage closure device system of claim 1, wherein the pre-curved portion forms a tip angle of between about 110 degrees and about 140 degrees.

6. The left atrial appendage closure device system of claim 5, wherein the pre-curved portion forms a tip angle of about 110 degrees.

7. The left atrial appendage closure device system of claim 1, wherein the bend radius is about 3.00 inches.

8. The left atrial appendage closure device system of claim 2, wherein the length of the body portion is about 2.72 inches.

9. The left atrial appendage closure device system of claim 1, wherein the access sheath has an outer diameter between about 10 F. and about 3 F.

10. The left atrial appendage closure device system of claim 9, wherein the access sheath has an outer diameter of about 14 F.

11. The left atrial appendage closure device system of claim 9, wherein the delivery sheath has an outer diameter about 2 F smaller than an outer diameter of the access sheath.

12. The left atrial appendage closure device system of claim 10, wherein the delivery sheath has an outer diameter of about 12 F.

13. The left atrial appendage closure device system of claim 1, wherein the left atrial appendage closure device is actuatable between a collapsed configuration and an expanded configuration.

14. The left atrial appendage closure device system of claim 13, wherein the left atrial appendage closure device is disposed within the delivery sheath in the collapsed configuration.

15. The left atrial appendage closure device system of claim 1, wherein the left atrial appendage closure device is releasably attached to a core wire slidably disposed within the delivery sheath.

16. A method of deploying a left atrial appendage closure device in a heart, comprising:
advancing an access sheath through a superior vena cava to a right atrium of the heart;
advancing a delivery sheath through the access sheath and into a left atrium of the heart; and
deploying a left atrial appendage closure device within an ostium of a left atrial appendage.

17. The method of deploying a left atrial appendage closure device of claim 16, wherein advancing the access sheath through the superior vena cava to the right atrium of the heart positions the access sheath through a wall of the heart between the right atrium and the left atrium and a distal tip of the access sheath proximate the ostium of the left atrial appendage.

18. The method of deploying a left atrial appendage closure device of claim 17, wherein advancing the delivery sheath through the access sheath and into the left atrium of the heart includes positioning a distal end of the delivery sheath within the ostium of the left atrial appendage.

19. The method of deploying a left atrial appendage closure device of claim 16, wherein the access sheath includes a pre-curved portion adjacent a distal tip of the access sheath, the pre-curved portion having a bend radius of about 3.00 inches.

20. A method of deploying a left atrial appendage closure device, comprising:
advancing a distal tip of an access sheath within a superior vena cava and into a right atrium of a heart;
positioning the distal tip of the access sheath within a left atrium of the heart proximate an ostium of a left atrial appendage;
advancing a distal end of a delivery sheath slidably disposed within the access sheath into the left atrium;
deploying a left atrial appendage closure device within the ostium of the left atrial appendage of the heart, the left atrial appendage closure device being releasably attached to a core wire slidably disposed within the delivery sheath;
expanding the left atrial appendage closure device into engagement with the ostium of the left atrial appendage; and
detaching the left atrial appendage closure device from the core wire.
